(19) United States
(12) Patent Application Publication
(10) Pub. No.: US 2010/0209966 A1
(43) Pub. Date: Aug. 19, 2010

(54) ASEPTIC BIOREACTOR SYSTEM FOR PROCESSING BIOLOGICAL MATERIALS

(75) Inventors: Keith Everett, Pittsboro, NC (US); Lynn F. Dickey, Cary, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Biolex Therapeutics, Inc.

(21) Appl. No.: 12/708,130

(22) Filed: Feb. 18, 2010

Related U.S. Application Data
(60) Provisional application No. 61/153,451, filed on Feb. 18, 2009.

(51) Int. Cl. C12P 1/00; C12M 1/00; C12M 1/38

(52) U.S. Cl. 435/41; 435/303.1; 435/286.1

(57) ABSTRACT

A bioreactor system according to one embodiment of the present invention is provided for producing and processing a biological material in an aseptic environment. For example, the system includes at least one culture tube configured to hold a liquid media containing a biological material and at least one flexible bag for promoting growth of the biological material therein. The liquid media and biological material are configured to be transferred from the culture tube to the flexible bag aseptically in an unclassified area. The system also includes at least one flexible harvest bag configured to be in flow communication with the flexible bag, wherein the flexible harvest bag is configured to separate biological material grown in the flexible bag from the liquid media aseptically in an unclassified area.

### CROSS-REFERENCE TO RELATED APPLICATIONS

The present application claims priority from U.S. Provisional Application No. 61/153,451 filed Feb. 18, 2009, the contents of which are incorporated herein by reference.

### BACKGROUND OF THE INVENTION

1. Field of the Invention

The present invention is related to a bioreactor system for processing biological material and, in particular, to a bioreactor system for producing and processing biopharmaceuticals in an aseptic environment.

2. Description of Related Art

Photo-bioreactors are devices that allow photosynthetic microorganisms to grow in a controlled manner. U.S. Pat. No. 5,846,816 to Forth (“Forth”) discloses a biomass production apparatus including a transparent chamber 10 which has an inverted, triangular cross-section, as is shown in FIG. 1 of Forth. Extending through the chamber is a first conduit 22 which has a plurality of perforations along its length to allow the introduction of gasses into the chamber. Also extending through the chamber are a pair of heat exchange conduits 26 connected to a supply of heat exchange medium.

The passage of air entering through the conduit establishes a distinctive flow pattern that causes the liquid in the chamber to circulate up through a central region of the chamber, across the upper portion of the chamber below a cover 16, and down along the chamber sidewalls 20 back to the conduit, as is shown in FIG. 3 of Forth. The cover includes two vents 28 through which the circulating gases exit the chamber. Ostensibly the passage of air and circulation of the liquid ensures that the biological matter suspended therein is exposed to light and also prevents the biological matter, such as algae, from adhering to the walls of the chamber.

Although the bioreactor disclosed by Forth promotes the growth of biological matter, it is generally not useful for applications requiring a sterile growth environment. The vents are open to external air which may include airborne contaminants. Such contaminants are especially troublesome for pharmacological applications wherein strict Food and Drug Administration guidelines for avoiding contamination must be met.

Moreover, conventional bioreactor systems typically require closed and classified areas for handling, growing, and harvesting the biological matter. These systems are expensive and generally require moving between classified areas, which may be time consuming and lead to inefficiencies. For example, cultures are typically transferred from plant bank storage tubes to downstream containers in a controlled environment, such as via a laminar flow hood. In addition, typical holding vessels for harvesting a biopharmaceutical are fitted with custom, closed liners with corresponding aseptic connectors that adapt to the tubing assemblies associated with the filter bag assembly. The tanks are bulky and expensive, are not disposable, and switching out liners aseptically may be time consuming and interrupt the process stream and connections between assemblies must be made under controlled environmental conditions to prevent microbial contamination.

Therefore, it would be advantageous to have a bioreactor and production system that is capable of aseptically producing and processing biopharmaceuticals without the need for controlled, aseptic production and processing suites. It would be further advantageous to provide a system that is inexpensive and capable of handling and promoting the growth of biological material and the production of biopharmaceuticals efficiently. Moreover, it would be advantageous to provide a system that is reliable, requires low maintenance, and provides a high production density.

### BRIEF SUMMARY OF THE INVENTION

Embodiments of the present invention may provide improvements over the prior art by, among other things, providing a bioreactor system for the closed and aseptic production and processing of a biopharmaceutical without the need of one or more classified, aseptic production and processing areas. According to one embodiment, the system includes at least one culture tube configured to hold an agar-based media or a liquid media containing a biological material and at least one flexible bag for promoting growth of the biological material therein. The culture tube and flexible bag are configured to facilitate transfer of the liquid media and biological material from the culture tube to the flexible bag aseptically in an unclassified area. The biological material produces a biopharmaceutical of interest and may or may not secrete the biopharmaceutical into the liquid media. The system also includes at least one flexible harvest bag configurable to be temporarily or constantly in flow communication with the flexible bag, wherein the flexible harvest bag is configured to separate biological material grown in the flexible bag from the liquid media to later facilitate the downstream processing of the biological material or media.

According to aspects of the system, the at least one culture tube and the at least one flexible bag include an aseptic connector configured to couple to one another and facilitate aseptic transfer of the biological material and liquid media. The at least one flexible bag may be a flexible seed bag or a flexible production bag. The system may include at least one flexible seed bag and at least one flexible production bag, wherein the at least one flexible production bag is configured to be in flow communication temporarily or continuously with the at least one flexible seed bag and the at least one flexible harvest bag. The at least one flexible bag may include a plurality of connection ports, wherein one of the ports is configured to couple with an over-pressure assembly. According to one aspect, a height of each of the at least one flexible bag is substantially less than a length and width thereof. The system may include a plurality of flexible bags. The plurality of flexible bags may be configured to be temporarily or continuously in flow communication with a single harvest bag. In addition, the system may include a support rack configured to support the plurality of flexible bags spaced apart vertically from one another. The system may further include at least one light source configured to illuminate the at least one flexible bag so as to promote growth of the biological material via photosynthesis.

An additional embodiment of the present invention is directed to a method of the production and processing of a biopharmaceutical in an aseptic environment. The method includes transferring a liquid media containing a biological material from at least one culture tube to at least one flexible bag and promoting growth of the biological material within the at least one flexible bag wherein growth of the biological material results in production and, in some embodiments, secretion of the biopharmaceutical into the liquid media. The method also includes separating the biological material grown in the at least one flexible bag from the liquid media, wherein each of the transferring, promoting, and harvesting steps occurs aseptically in an unclassified area.

Aspects of the method include exposing the at least one flexible bag to a light source so as to promote growth of the biological material via photosynthesis. The promoting step may include promoting growth of the biological material within at least one flexible bag such that the biological material produces and secretes a biopharmaceutical into the liquid media. The method may further include aseptically transferring the biological material and liquid media from the at least one flexible bag to at least one harvest bag, wherein separating comprises filtering the biological material from the liquid media in the at least one harvest bag. The transferring step may include temporarily or permanently coupling the at least one culture tube and the at least one flexible bag with respective aseptic connectors. Moreover, the method may include positioning a plurality of the flexible bags in a support rack spaced apart vertically from one another and/or automatically measuring and controlling a temperature of the biological material in the at least one flexible bag.

Another embodiment of the present invention is directed to a flexible harvest bag assembly for harvesting a biological material and/or at least one biopharmaceutical aseptically in an unclassified area. The flexible harvest bag assembly may be disposable. The flexible harvest bag assembly includes a flexible bag (e.g., a pair of outer layers of flexible material coupled to one another) defining an enclosure therein. The flexible harvest bag assembly further includes an inlet defined in the flexible bag and configured to receive a solid biological material and a liquid media and to direct the biological material and liquid media into the enclosure. The flexible harvest bag assembly also includes a filter positioned within the enclosure, wherein the filter is configured to separate at least a portion of the solid biological material from the liquid media. In addition, the flexible harvest bag assembly includes an outlet defined in the flexible bag that is configured to transfer the filtered liquid media out of the enclosure while the solid biological material remains within the enclosure. Where the biological material has expressed or produced a biopharmaceutical that is not secreted, the biological material may be collected for further processing. Alternatively, where the biological material has expressed or produced a biological material and/or a biopharmaceutical that is secreted into the liquid media, the liquid media may be collected for further separation or isolation of the biological material and/or biopharmaceutical.

Aspects of the flexible harvest bag assembly include a flexible bag comprising a pair of outer layers of flexible material coupled to one another to define an enclosure therebetween. The pair of outer layers may be an expandable polymeric material. The inlet and the outlet may be defined in one of the outer layers of flexible material and in one aspect, the inlet and outlet are defined in the same outer layer. The inlet and outlet may be located at opposite ends of the same outer layer of flexible material. The harvest bag may further include an air release valve defined opposite the inlet in the other outer layer of flexible material. In addition, the filter may be positioned within the enclosure and between the outer layers. One of the outer layers may be secured to the filter to define a first pocket within the enclosure for retaining the solid biological material therein, and the outer layers may be secured together to define a second pocket within the enclosure for retaining the liquid media therein. According to one aspect, the second pocket includes a hold reservoir for buffering inconsistencies in flow rate through the inlet and the outlet due to harvesting conditions without causing over-pressure within the flexible bag. Furthermore, the outer layers may be secured together to define an opening for receiving a support rod therethrough, wherein the support rod is configured to support the flexible bag vertically such that the biological material and the liquid media are capable of entering the inlet in an upper portion of the one of the outer layers and the liquid media is capable of exiting the outlet in a lower portion of one of the outer layers. The flexible bag may have a generally rectangular shape proximate the inlet and a generally triangular shape proximate the outlet.

An additional embodiment of the present invention is directed to a support rack for supporting a plurality of flexible bags and promoting growth of a biological material in a liquid media. The support rack includes a plurality of upright support members and a plurality of laterally-extending support rails interconnecting the upright support members. The support rack further includes a plurality of shelves operably engaged with the laterally-extending support rails and being configured to support the plurality of flexible bags spaced apart vertically from one another. The support rack also includes a feedback control system for automatically measuring and controlling a temperature of the biological material in the plurality of flexible bags.

According to one aspect of the support rack, the shelves are slidably engaged with the laterally-extending support rails and configured to be moved between a stowed position and an extended position relative to the upright support members. The support rack may include at least one light source for illuminating the plurality of flexible bags and/or an air circulating device operably engaged with the support rack for circulating an air supply around the at least one light source. The support rack may alternatively include a plurality of light sources disposed between the plurality of shelves. Each of the shelves may be substantially transparent and/or include a corrugated polycarbonate material.

A further embodiment is directed to a culture tube assembly. The assembly includes a culture tube configured to hold an agar-based media or a liquid media containing a biological material and an aseptic connector assembly coupled to the culture tube and configured to aseptically contain the media and biological material within the culture tube. The aseptic connector assembly includes tubing for coupling with the culture tube, as well as an aseptic connector configured to transfer the liquid media containing the biological material aseptically in an unclassified area to at least one flexible bag for promoting growth of the biological material.

### BRIEF DESCRIPTION OF THE SEVERAL VIEWS OF THE DRAWINGS

Having thus described the invention in general terms, reference will now be made to the accompanying drawings, which are not necessarily drawn to scale, and wherein:

FIGS. 1a-1f illustrate a bioreactor-based production and harvest system according to one embodiment of the present invention;

FIG. 2 is a perspective view of a culture tube assembly and an aseptic connector assembly according to one embodiment of the present invention;

FIG. 3 is another perspective view of the aseptic connector assembly shown in FIG. 2;

FIG. 4 is perspective view of an array of culture tube and aseptic connector assemblies shown in FIG. 1 according to one embodiment of the present invention;

FIGS. 5a-5d illustrate various views of the aseptic connector assembly shown in FIG. 2;

FIG. 6 is a perspective view of a flexible seed bag according to one embodiment of the present invention;

FIGS. 7 and 8 are additional perspective views of the flexible seed bag shown in FIG. 6;

FIG. 9 is a perspective view of a flexible production bag according to one embodiment of the present invention;

FIG. 10 is an elevation view of the flexible production bag shown in FIG. 9;

FIG. 11 is a perspective view of a support rack configured to support the flexible seed bag shown in FIGS. 6-8 and the flexible production bag shown in FIGS. 9 and 10 according to an additional embodiment of the present invention;

FIG. 11a is an enlarged view of the support rack shown in FIG. 11 illustrating a feedback control system;

FIG. 12 is a perspective view of a support rack shown in FIG. 11 in use according to one embodiment of the present invention;

FIGS. 13a-e show various views of the support rack shown in FIG. 11;

FIGS. 14a-d depict various views of a cassette configured for use with the support rack shown in FIG. 11 according to one embodiment of the present invention;

FIG. 15 is an elevation view of a flexible harvest bag according to an additional embodiment of the present invention;

FIG. 16 is an elevation view of the flexible harvest bag shown in FIG. 15 in use according to an embodiment of the present invention;

FIGS. 17a-b are elevation and perspective views of the flexible harvest bag shown in FIG. 15;

FIGS. 18a-g are various views of the flexible harvest bag shown in FIG. 15;

FIGS. 19a-d illustrate various views of a flexible harvest bag according to another embodiment of the present invention;

FIGS. 20a-d depict a support rack and harvest bag cart according to one embodiment of the present invention;

FIG. 21 is a perspective view of a harness assembly according to an embodiment of the present invention; and

FIGS. 22a-g are views of the harness assembly shown in FIG. 21.

### DETAILED DESCRIPTION OF THE INVENTION

Various embodiments of the present invention now will be described more fully hereinafter with reference to the accompanying drawings, in which some, but not all embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like numbers refer to like elements throughout. The singular forms “a,” “an,” and “the” include plural referents unless the context clearly dictates otherwise.

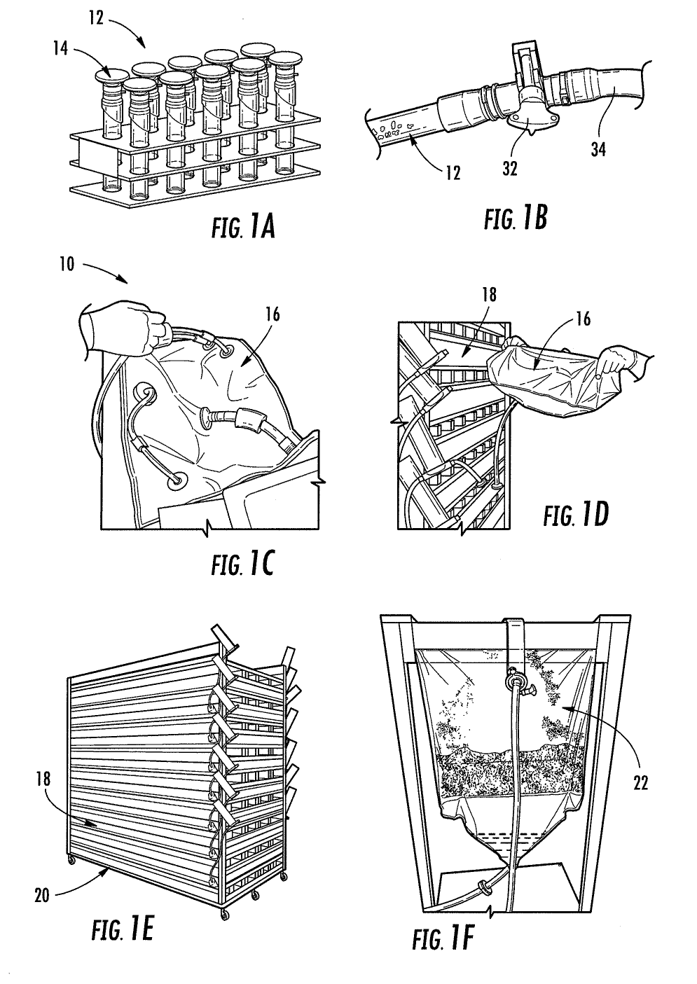

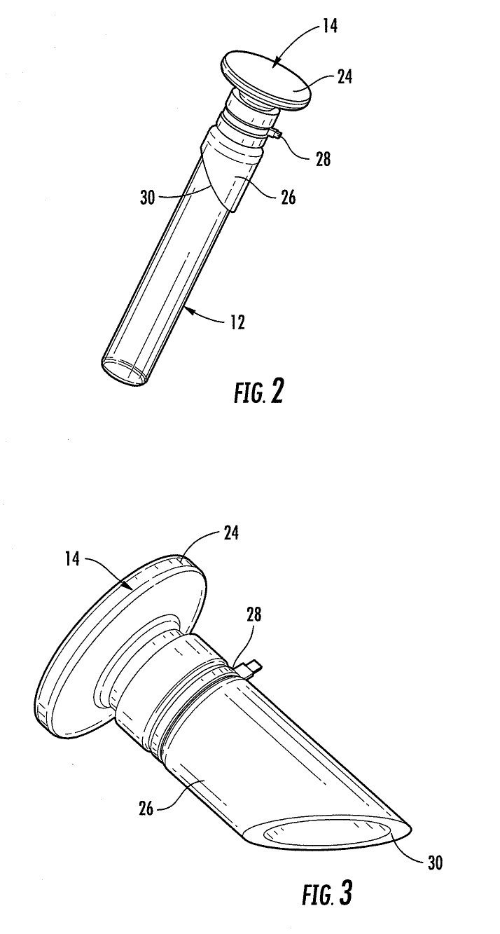

A bioreactor system of one embodiment of the present invention is generally shown in FIGS. 1a-f. Included in the bioreactor system 10 are a plurality of culture tubes 12 each having an aseptic connector assembly 14 as shown in FIGS. 1a and 1b. The culture tubes 12 are configured to aseptically transfer biological material contained within a liquid media downstream to a flexible seed bag 16 as shown in FIGS. 1b and 1c. The flexible seed bags 16 are configured to promote growth of the biological material therein. Moreover, the system 10 includes flexible production bags 18 that are capable of aseptically receiving biological material and liquid media from the flexible seed bags as shown in FIG. 1d. The flexible seed bags 16 and production bags 18 may be supported by a support rack 20 in a vertical stack as shown in FIG. 1e. Furthermore, the system 10 includes flexible harvest bags 22 that are capable of receiving biological material and liquid media from the production bags and separating the biological material from the liquid media. As will be explained in further detail below, each of the steps involved in transferring and processing the biological material and liquid media is carried out aseptically and in an unclassified area, and each of the system components in contact with the biological material and media may be disposable.

The term “media” as used herein refers to any liquid, gel, partially liquid-partially solid, or otherwise flowable supply of compounds, chemicals or nutrients that are used to promote the growth, testing, modification or manipulation of the biological matter housed within the flexible seed bags 16 and production bags 18. Media therefore, can be water alone, a combination of water with fertilizer, nutrients, vitamins, growth factors, hormones, soil, an agar gel, mud or other combination of components, with or without water, as long as some type of flow and manipulation of the components can be induced using the devices described herein. After growth of the biological materials, the liquid media may also include biological material or biopharmaceuticals as a result of the growth of the biological material therein such that the media may be the end product desired for downstream processing.

The term “biological materials” or “biological matter” as used herein describe any material that requires a supply of media in order to support proliferation or pharmaceutical compound expression. The biological materials may be phototropic or non-phototropic material that requires aeration and/or exposure to artificial or natural light. Preferably, the biological materials are aquaculture adaptable species or aquatic plants that require or thrive on liquid surfaces, such as plants within the duckweed family Lemnaceae (“Lemna”) or algae. Other aquatic plants include Giant Salvinia, Kariba weed, Aquarium watermoss, Water Fern, Carolina mosquito fern, water hyacinth, jacinthe d'eau, Variable-leaf Pondweed, Waterthread Pondweed, Hydrilla, American Water-Plantain, Marsh Pennywort, and Creeping Rush as well as a range of terrestrial plants that are adaptable to aquaculture. These plants and other biological material may be either wild plants, or transgenic plants for the production of vaccines, therapeutic proteins and peptides for human or animal use, neutraceuticals, industrial process additives, small molecule pharmaceuticals, research and production reagents (growth factors and media additives for cell culture) or excipients for pharmaceuticals. Biological materials may also include other cells useful for the production of a protein of interest including but not limited to yeast, mammalian cells, and microorganisms.

Moreover, the media may also include biological material as described above. For example, the media may include biological material having both a solid phase and a liquid phase. In one embodiment, the “liquid phase” may be separated from the suspended and accompanying “solid phase” of the liquid media using a filter (e.g., using flexible harvest bags 22). The solid phase may include plants, parts of plants, detritus from the culture, and any particles suspended in the media, larger than the pore size of the filter material, whereas the liquid phase may include minor non-dissolved contaminants and the dissolved components left in the media after the culture has used the media, as well as those dissolved components added to the media by the plant culture, including, in some cases, biological compound(s) of interest.

The term “biopharmaceutical” is intended to include a biological or chemical product produced by the biological material. Such biopharmaceuticals include hormones, blood factors, thrombolytics, vaccines, interferons, monoclonal antibodies, therapeutic enzymes, chemical entities, and the like.

FIGS. 2-5 illustrate a culture tube 12 and aseptic connector assembly 14 according to one embodiment of the present invention. The culture tube 12 or slant tube may be any suitable tube or vessel that is configured to hold a liquid media containing a biological material therein. For example, the biological material may be fronds contained within a liquid media. The aseptic connector assembly 14 includes an aseptic connector 24 or connector coupled to a tubing 26 such as via a wire tie 28 or other suitable connection. According to one embodiment the aseptic connector 24 is a BioQuate™ aseptic connector manufactured by BioQuate Inc. of Clearwater, Fla. The aseptic connector 24 may have a barb fitting or the like that is configured to engage the tubing 28 thereby sealing the biological material and liquid media aseptically within the culture tube 12. The end 30 of the tubing 26 opposite the aseptic connector 24 may include a diagonal cut as shown in FIG. 3, which may be used to facilitate insertion of the culture tube 12 within the tubing where the tubing and culture tube are coupled in a press fit.

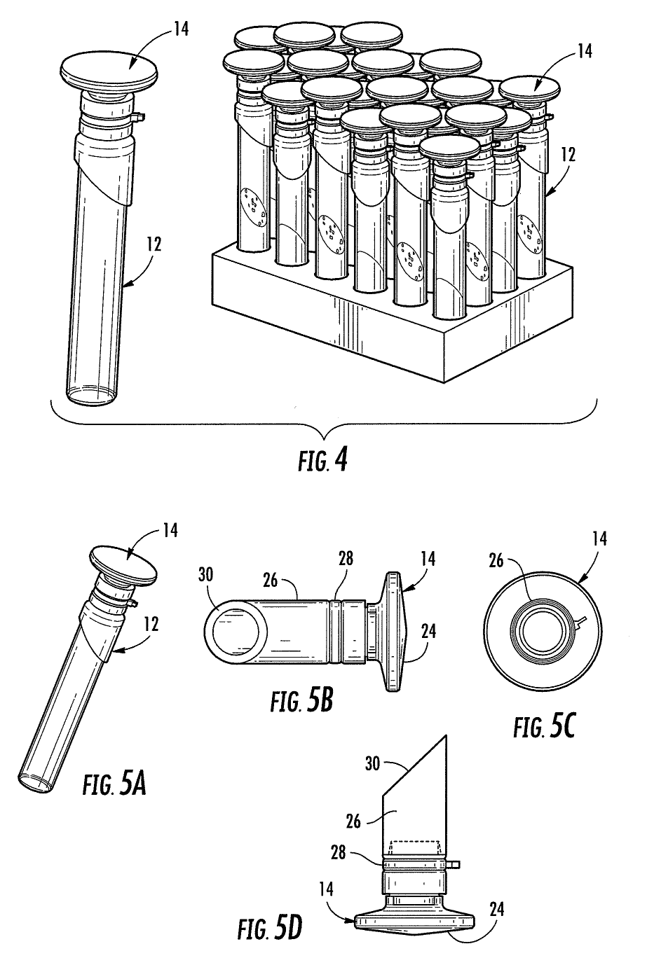

As known to those of ordinary skill in the art, the aseptic connector assembly 14, including all contact surfaces, are typically in compliance with current United States Pharmacopeia (USP), good manufacturing processes (GMP), and Food and Drug Administration (FDA) requirements in order to ensure that aseptic conditions are maintained. Moreover, the materials of the aseptic connector assembly 24 and tubing 26 typically have the requisite material properties for sterilization (e.g., UV stable) and are typically free of toxins, extractables/leachables, or other agents that may affect the aseptic properties of the materials. The culture tubes 12 may be various sizes depending on the particular biological material being transferred and the size of the flexible bag 16, 18 being transferred to. According to one embodiment, the tubing 26 has an inner diameter of about ⅝ of an inch or larger, and the end 30 may be formed at an angle of about 45°, wherein the length of the tubing at its longest point is about 60-70 mm and at its shortest point is about 35-45 mm (see FIG. 5d).

According to one embodiment, a plurality of culture tubes 12 may be maintained in a master plant bank for subsequent inoculation. For example, the culture tubes 12 may contain a liquid media, such as agar, and biological material, such as Lemna, and be stored as the master plant bank under controlled conditions for extended periods of time, typically in a state of “stasis”, (e.g., up to 2 years) before being used for inoculation. Moreover, the culture tubes 12 may be used to periodically subculture or multiply the Lemna for preparing additional culture tubes for subsequent inoculation. For instance, the Lemna may be aseptically removed from the culture tubes 12 and placed in bottles and grown in an incubator. The Lemna may then be transferred aseptically from the bottles into fresh culture tubes 12 and placed in the master plant bank until needed for inoculation.

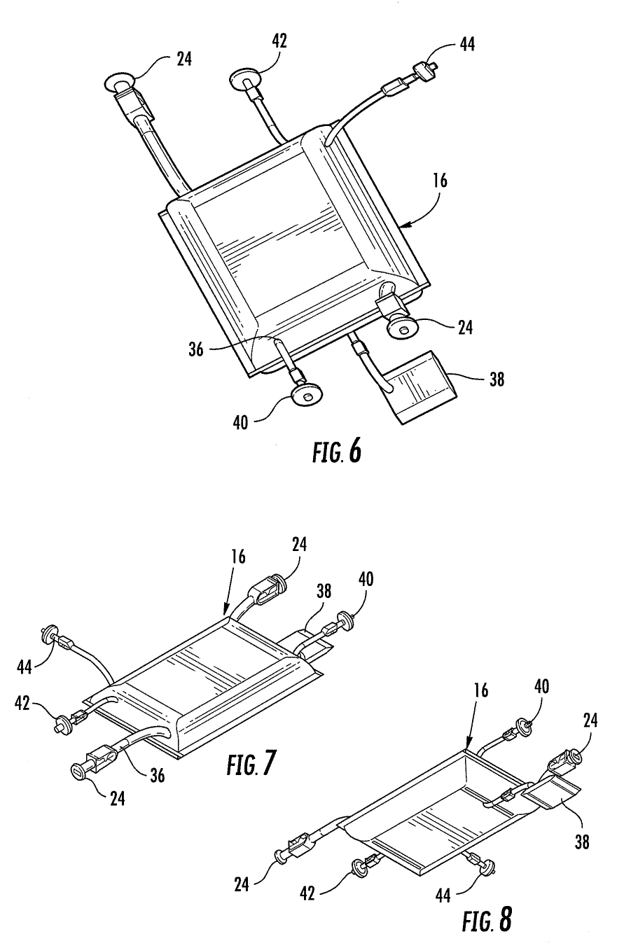

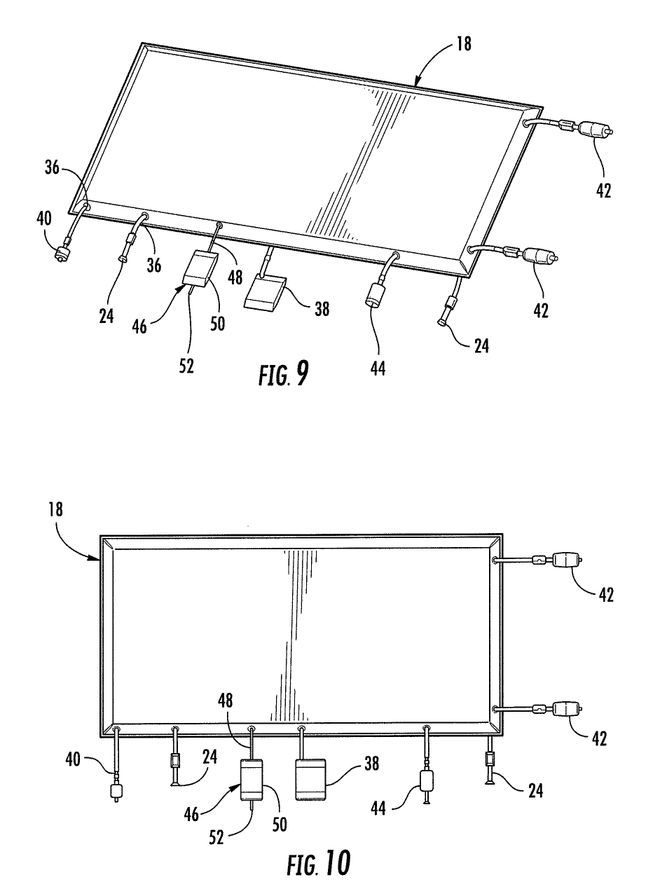

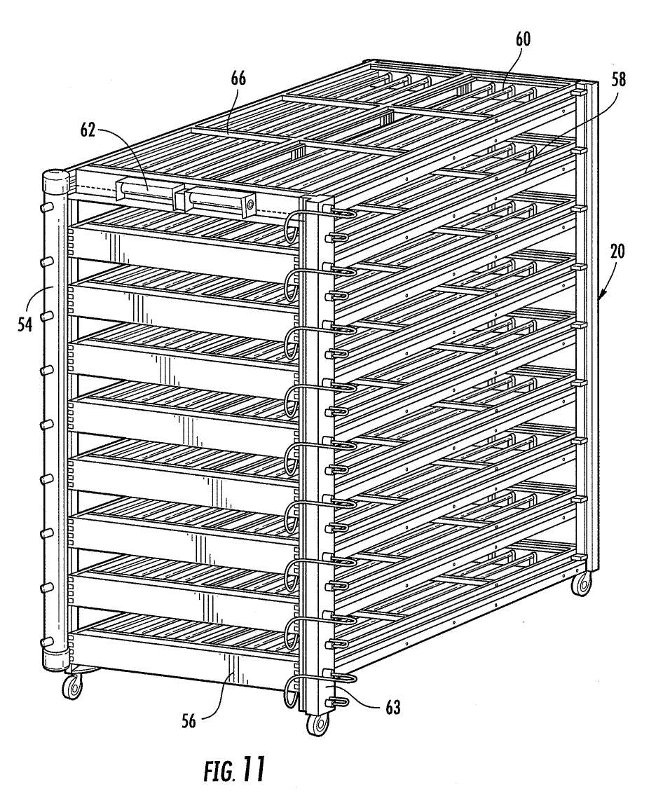

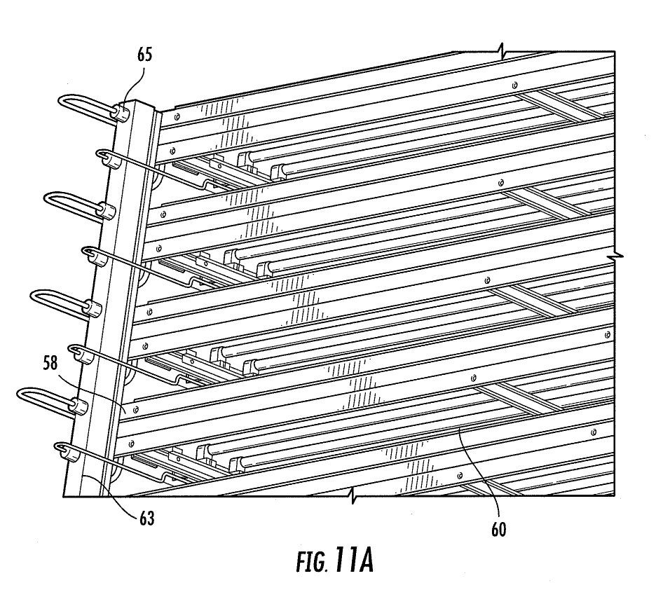

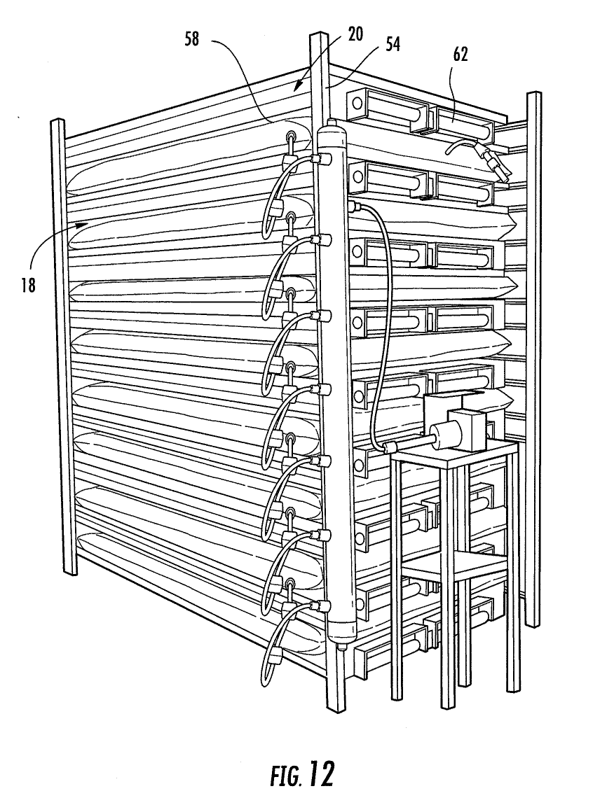

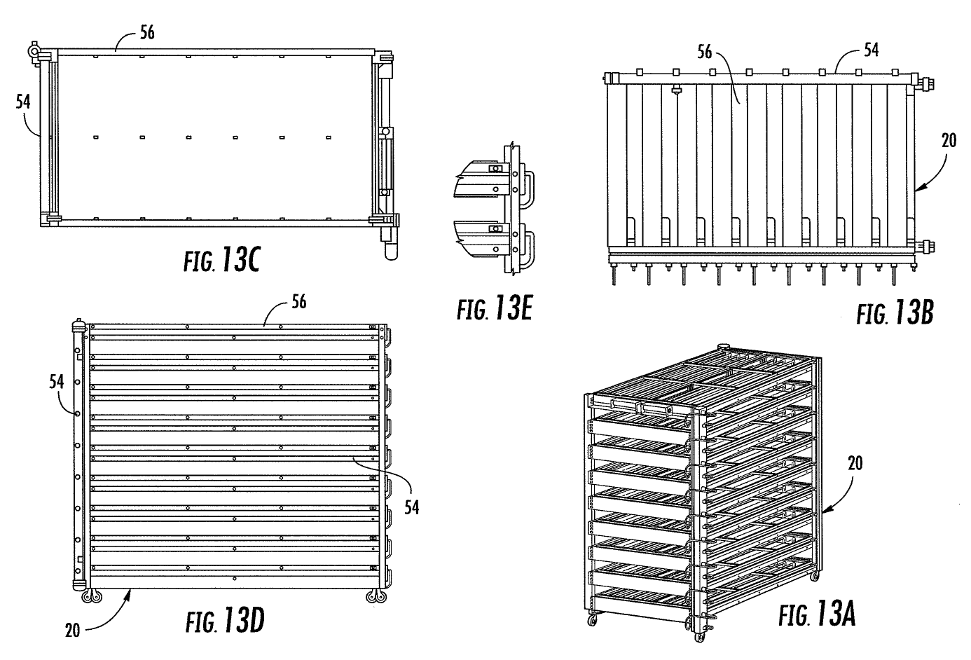

The flexible seed bags 16 may include a corresponding aseptic connector 24 configured to couple with the aseptic connector 24 of the culture tube 12 with a strain relief clamp 32 or similar clamp (see FIGS. 1b, 6, and 7). The connection of the aseptic connectors 24 allows liquid media and biological material within the culture tube 12 to be transferred to the flexible seed bag 16 aseptically and in an unclassified area. According to one embodiment, once the connection between the culture tube 12 and flexible seed bag 16 has been made and the transfer of biological material and liquid media is complete, a heat sealer or pinch clamp can seal the culture tube 12 and transfer tube 34, and the culture tube and transfer tube can be cut away leaving the terminated and sealed end of the transfer tube attached to the flexible seed bag. Thus, the biological material and liquid media may be quickly transferred to the flexible seed bags 26, and the culture tubes 12 may be disposed of once the transfer tube 34 is heat-sealed or clamped. Although the connection between the culture tube 12 and flexible seed bag 16 may be temporary, the connection may be permanent if desired.

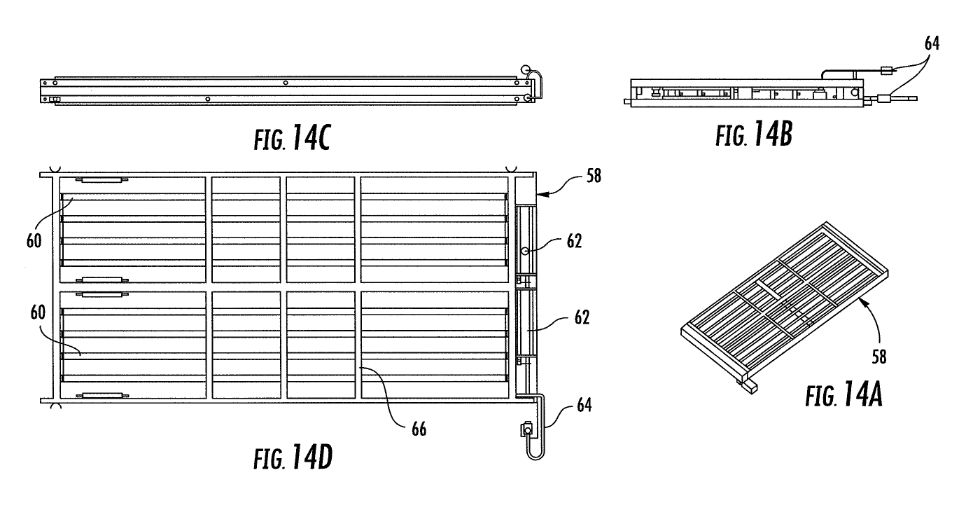

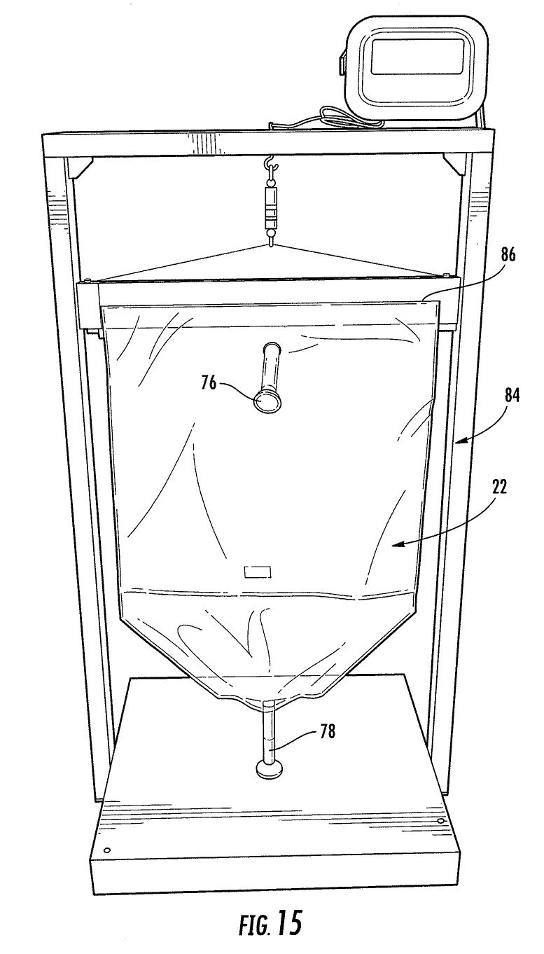

FIGS. 6-8 illustrate a flexible seed bag 16 according to one embodiment of the present invention. The flexible seed bags 16 may be employed to receive biological material and liquid media from the culture tubes 12 as described above. In addition, FIGS. 9 and 10 depict a flexible production bag 18 according to one embodiment of the present invention. The flexible production bags 18 are capable of receiving biological material and liquid or agar-based media from the flexible seed bags 16 aseptically in an unclassified area. According to one embodiment, the flexible production bag 18 may have an aseptic connector 24 or the like that is configured to connect to the aseptic connector 24 of the flexible seed bag 16 via inoculation ports 36 in order to aseptically transfer biological material and liquid media therebetween. It is understood that the connection between the flexible seed bag 16 and flexible production bag 18 may be temporary or permanent such that the flexible bags 16, 18 may be in temporary or continuous flow communication with one another.

Although flexible seed bags 16 have been described, it is understood that the biological material may be transferred directly from the culture tube 12 to the production bag 18. Thus, in one embodiment, the seed bags 16 may be optional such that the production bags 18 may be inoculated with the biological material contained in the culture tubes 12 via aseptic connectors 24.

The flexible bags 16, 18 may be constructed of a light transmissive material which allows the passage of sufficient light to promote growth of the biological material, and production of a biopharmaceutical of interest, stored therein. For instance, the flexible bags 16, 18 may be constructed of a polymeric material, such as a polycarbonate, polyvinylchloride, polystyrene, TEFLON, silicone, nylon, polyethylene, or any FDA-approved polymer material. In some embodiments, these materials may be flexible as shown generally in FIGS. 1c and 1d. The flexible bags 16, 18 may be capable of being partially filled with media so as to create a media surface on which the biological material is supported. According to one embodiment, the flexible bags 16, 18 are formed with two pieces of material joined together about their periphery to form a two-dimensional bag with no gussets (e.g., joined with heat sealing). Thus, the flexible bags 16, 18 may be formed as “pillow bags” that may be cost efficient and simple to manufacture.

Furthermore, the liquid media may serve to at least partially inflate the flexible bags 16, 18 such that the flexible bags 16, 18 are substantially self-supporting when partially filled with media. Furthermore, according to some embodiments, the flexible bags 16, 18 may comprise a substantially gas-permeable polymer membrane such that gasses may be introduced into and/or vented therethrough without the use of a nozzle (as described below). According to another embodiment, it should be understood that the flexible bags 16, 18 described herein may serve as a disposable “liner” for one or more substantially rigid containers, such as those disclosed in U.S. Patent Appl. Publ. No. 2007/0113474, which is hereby incorporated herein by reference in its entirety.

The use of flexible bags 16, 18 to define an aseptic reservoir may, in some embodiments, provide a relatively inexpensive and flexible system 10 for supporting the growth of biological material (such as aquatic plant material) in bulk. The use of disposable flexible bags 16, 18 may also reduce and/or eliminate the need for costly and time-consuming re-qualification and/or validation of reusable containers (such as cylindrical “pipes” or tanks) that may be required if the system 10 is used to produce and/or support biological materials in a GMP setting.

As shown generally in FIGS. 6-10, the overall shape of the flexible bags 16, 18 may be chosen to maximize the surface area that is capable of supporting growth of a biological material. Furthermore, the flexible bags 16, 18 may be provided with a substantially constant cross-section along their length. For example, the cross-section of the flexible bags 16, 18 may be substantially rectangular as shown generally in FIGS. 6-10. FIGS. 6-10 also demonstrate that the flexible seed bags 16 may be smaller in dimension than the flexible production bags 18. For example, the flexible seed bags 16 may be about half of the size of the flexible production bags 18. According to one exemplary embodiment, the flexible seed bag 16 has a length of about 2 feet and a width of about 2 feet, while the flexible production bag 18 has a length of about 8 feet and a width of about 4 feet.

However, it should be recognized that any size and configuration of flexible bags 16, 18 may be used as long as a proportionately large media surface can be provided for the growth of biological materials (e.g., the flexible seed and production bags could be the same size). For example, the flexible production bags 18 could have a length of about 16 feet and a width of about 8 feet. Other shapes could also be used for the flexible bags 16, 18 including shapes with, and without, a constant cross-section. For instance the flexible bags 16, 18 may have a round or oval shape, or some arbitrary or irregular shape constructed to fit lighting needs or available space. Preferably, however, the shape is chosen to maximize the surface area of the portion of a cross-section of the flexible bags 16, 18 in a plane that is orthogonal to the pull of gravity (i.e., a horizontal plane). While the flexible bags 16, 18 may be of any shape, a substantially rectangular shaped flexible bag may be especially beneficial for providing a relatively low aspect ratio, which may, in turn, create beneficial non-laminar, turbulent, or chaotic flow as gasses are introduced and/or removed from the flexible bag. Such a low aspect ratio may thus prevent and/or minimize the production of laminar flow zones within the flexible bags 16, 18 that may isolate fresh gases from gas-depleted areas (which may result in depletion zones where growth of the biological material may be discouraged and/or inhibited). For example, in some embodiments, the flexible bags 16, 18 may have a ratio of length to width of less than about 2. For a further discussion of exemplary sizes and configurations of a flexible bag, see U.S. Patent Appl. Publ. No. 2007/0113474, which is hereby incorporated herein by reference in its entirety.

The flexible bags 16, 18 may further comprise at least one opening 36 or port defined therein for allowing limited and aseptic access pathways into the flexible bags 16, 18. For example, the openings 36 defined by the flexible bags 16, 18 may allow for the insertion and securing of a variety of devices that may include, but are not limited to: a sampling bag 38, a gas inlet assembly 40, a gas exit assembly 42, and a media inlet assembly 44. It should be noted that other measurements within the flexible bags 16, 18 could also be made with a variety of other devices depending upon the information desired by the user. For example, and as shown generally in FIGS. 9 and 10, the flexible production bag 18 may include an over-pressure assembly 46 for ensuring that the pressure within the flexible production bags does not exceed a predetermined pressure, which may prevent the flexible production bags from exploding. The over-pressure assembly 46 may employ check valves 48, 52 and an indicator 50 that are capable of releasing pressure in the flexible production bag 18 if a predetermined pressure is reached (e.g., about 0.07 PSI). For example, the over-pressure assembly 46 may include a first check valve 48 coupled to the flexible production bag 18 and an indicator 50 (e.g., a bladder) that is coupled to a second check valve 52. The first check valve 48 is configured to open when the predetermined pressure within the flexible production bag 18 is reached resulting in the bladder 50 filling with gas. When the pressure within the bladder 50 reaches the predetermined pressure, the second check valve 52 opens and releases air from the bladder to keep the bladder and flexible production bag 18 from bursting.

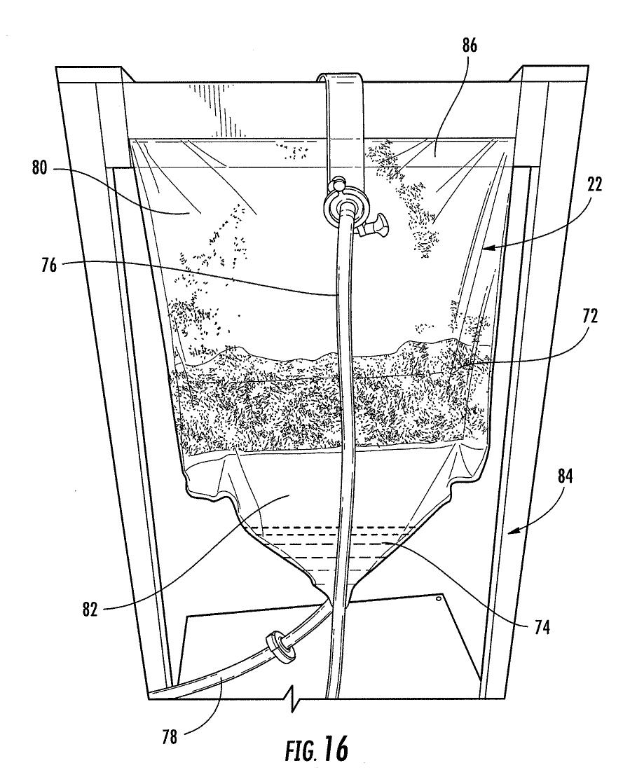

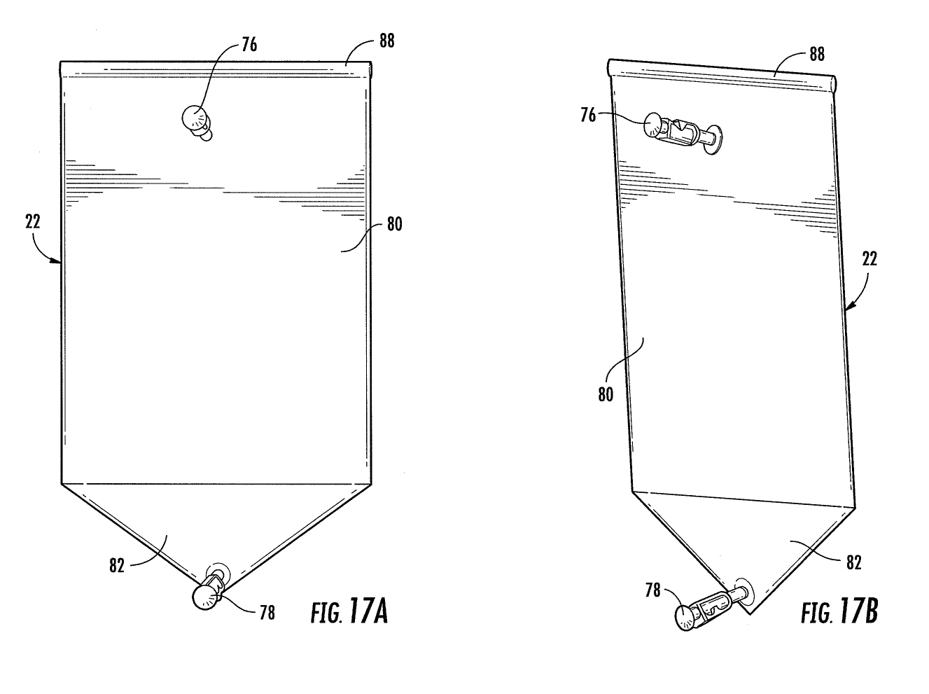

As shown in FIGS. 11-13, the system 10 may employ a support rack 20 for supporting a plurality of flexible bags 16, 18 and promoting growth of a biological material in a liquid media. For example, as shown generally in FIG. 12, the support rack 20 may support a plurality of flexible production bags 18 in a vertical stack spaced apart from one another. The support rack 20 may include a plurality of upright support members 54, a plurality of laterally extending support rails 56 interconnecting the upright support members, and a plurality of shelves 58. As shown in FIGS. 11-13, the shelves 58 may be operably engaged with the laterally-extending support rails 56 and configured to carry the flexible bags 16, 18 in a substantially vertical stack. The shelves 58 may each comprise a substantially transparent enclosure configured to be capable of enclosing and/or carrying the flexible bags 16, 18 and the one or more light sources 60 (which may, in some embodiments, comprise a plurality of elongate fluorescent tubes disposed within the shelves). Thus, the shelves 58 may be configured to illuminate both a top (via the light source 60 carried by a shelf disposed vertically above the flexible bag 16, 18) and bottom (via a light source carried by the shelf on which the flexible bag is supported) side of the flexible bag so as to encourage and/or facilitate the growth of an aquatic plant that may be suspended therein.

According to one embodiment, the support rack 20 may further include an air circulating device 62 for circulating an air supply around the flexible bags 16, 18 for controlling a temperate within the flexible bags and removing excess heat before it is transferred to the flexible bags. The air circulating device 62 may include, but is not limited to: a blower, a ducted fan, an air conditioning device, a box fan, or the like. Furthermore, in some embodiments, wherein the support rack 20 comprises one or more shelves 58 (and wherein each shelf includes a substantially transparent enclosure configured to be capable of enclosing and/or carrying one or more light sources 60), the air circulating device 62 may be operably engaged with the shelf so as to be capable of circulating an air supply around the light source carried by the shelves so as to dissipate heat and/or otherwise cool the shelves such that the temperature within the flexible bags 16, 18 carried by the shelves may remain relatively constant over time (even in cases where the light sources are illuminated for long periods of time). According to one embodiment, the shelves 58 may be fitted with “active” cooling elements where cooled liquid may be passed through coils adjacent to the shelves for cooling the shelves.

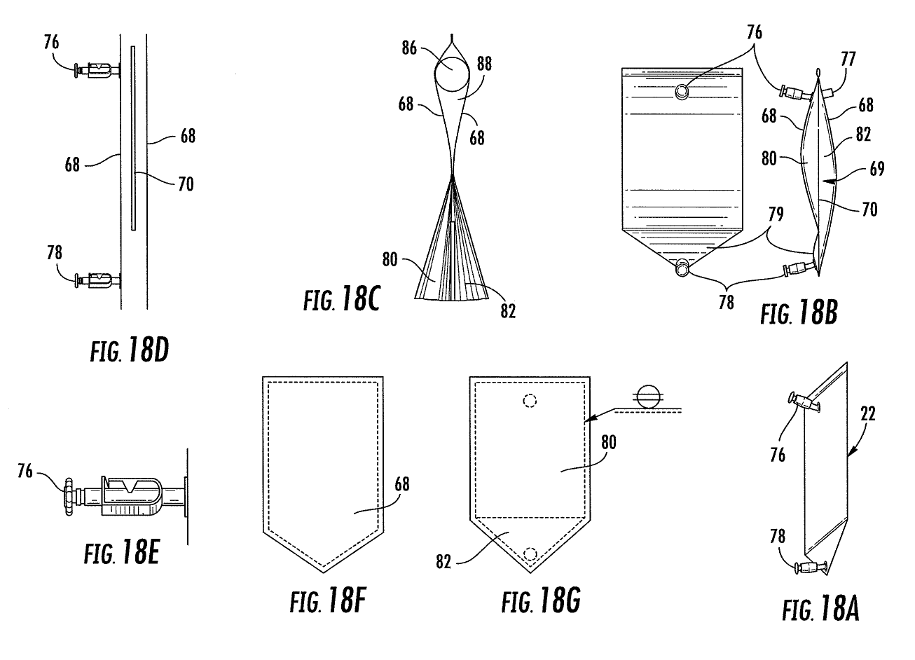

The shelves 58 may be slidably disposed within the support rack 20 such that the shelves may be extended laterally from the stack formed by the support rack and such that the flexible bags 16, 18 carried by the shelves may be accessible for maintenance and/or replacement when the shelves are extended relative to the support rack. For example, FIG. 14 illustrates that the shelves 58 may be removable cassettes that are configured to slidably engage the laterally-extending support rails 56 of the support rack 20 (e.g., via one or more bearing tracks that may be operably engaged with the laterally-extending support rails) such that the shelves may be moved between a stowed position and an extended position relative to the upright support members 54. Thus, according to some such embodiments, the flexible bags 16, 18 carried by the shelves 58 may be substantially accessible when the shelves are disposed in the extended position. Such embodiments, may also allow a user to more easily access the light source 60 that may be carried by the shelves 58. However, it is understood that the shelves 58 may be removable (see FIG. 14) or fixedly attached to the support frame 20. The support rack 20 incorporating fixed shelves 58 may be more cost efficient and lighter weight, while the removable shelf or cassette may be easily serviced. According to one embodiment shown in FIG. 14, the shelves 58 or cassettes are formed from a corrugated, clear polycarbonate barrier material supported by a frame 66, as well as air circulating device 62, light sources 60, and connections 64 for the same. The corrugated plastic barrier material may provide support for the flexible bags 16, 18, insulate against heat, and allow passage of diffused light for promoting growth of the biological material.

As shown in FIG. 12, the flexible bags 16, 18 may be positioned adjacent a light source 60 (such as one or more lights) for illuminating the flexible bags and the media surface created therein on which the biological material may be supported. According to some such embodiments, the light source 60 may be positioned substantially parallel to each of the plurality of flexible bags 16, 18 and may be disposed substantially within the spacing between the flexible bags. For example, FIGS. 11 and 14 demonstrate that the light sources 60 may be integrated with each shelf 58 or cassette.

The light sources 60 may be artificial lights that are electrically powered. For instance, lighting can be supplied by light-emitting diodes, neon, fluorescent lights, incandescent lights, sodium vapor lights, metal halide lights, or various combinations of these, and other, types of lights. Alternatively, the artificial lights may also be aided by, or replaced with, direct and indirect sunlight. However, artificial lights are preferred due to their ease of control and positioning so that all of the duckweed, or other biological material, contained in the flexible bags 16, 18 is supplied a sufficient amount of light to promote growth. Supplying power to the various types of light sources can be done via wiring, or other manner that is conventional in the art and therefore not described herein in additional detail.

According to one embodiment, each shelf 58 is configured to support four flexible seed bags 16 (e.g., a total of 32 flexible seed bags for an 8-shelf support rack 20), while in another embodiment, each shelf is configured to support a single flexible production bag 18 (e.g., a total of 8 flexible seed bags for an 8-shelf support rack). In one aspect, a 4×8×8 foot, 8-shelf support rack 20 provides 100 cubic feet of lit production volume, wherein each shelf 58 is within 1-5″ of the light source 60, so by virtue of Beer's law there may be more lighting of the culture with less overall intensity needed because the light travels less distance (light intensity falls inversely to the square of the distance). So fewer bulbs and heat management may be needed, and costs may be lowered per cubic foot of culture space. An 8×16 foot support rack 20 may provide 380 cubic feet of culture volume with only a 132 sq ft footprint.

It should also be noted that the relative positions and number of the light sources 60 and the flexible bags 16, 18 may be modified to suit a particular application. For instance, larger numbers of light sources 60 could be used to accelerate growth of the biological material, or larger numbers of flexible bags 16, 18 stacked in a tighter arrangement on the support rack 20 may be used to grow larger amounts of biological material (e.g., about 3 to 6 inches of depth between shelves 58). Therefore, the combinations of light sources and flexible bags 16, 18 are not necessarily restricted to the above-listed configurations and would still fall within the scope of the present invention. For additional discussion regarding an exemplary support rack, see U.S. Patent Appl. Publ. No. 2007/0113474, which is hereby incorporated herein by reference in its entirety.

The support rack 20 may also employ a feedback control system 63 for automatically measuring and controlling a temperature of the biological material in the plurality of flexible bags 16, 18 (see FIGS. 11 and 11a). The feedback control system 63 may employ a power strip having a plurality of ports 65 (e.g., for temperature sensors, air circulating units, or the like) or a single port accommodating a separate multi-port multiplexer, as well as software control of their power outlets. Temperature sensors in contact with the flexible bags 16, 18 may be used to protect the biological material via the feedback control aspect of the power strips. According to one embodiment, the power strip is a Pulizzi Intelligent Power Control product manufactured by Eaton Corporation of Santa Ana, Calif.

According to one aspect, the temperature of the culture within the flexible bags 16, 18 may be monitored by a temperature probe in contact with a representative flexible bag, which is read by the power strip. The power strip is configured with a software algorithm that will turn off the light source 60 (heat source) if the temperature in the flexible bags 16, 18 goes above a predetermined upper temperature limit. In this way, if there is a failure in the growth room HVAC or an air circulating unit 62 causing the culture temperatures to rise, the control system will turn off the light source on the support rack 20 and thereby eliminate the major heat source on the support rack. In contrast, if during the cold months, the HVAC heating system fails and the ambient temperature around the culture becomes too cold, the control system may sense the drop in culture temperature and turn off all the air circulating units 62 on the support rack 20. Thus, heat from the light source 60 will build up the temperature in the flexible bags 16, 18 and keep the cultures at the proper temperature. As the temperature in the flexible bags 16, 18 rises back up to normal, the moderated temperature will be sensed and the air circulating units 62 may turn back on as needed to keep the temperature from rising too high.

FIGS. 15-18 illustrate a flexible harvest bag 22 according to one embodiment of the present invention. The flexible harvest bag 22 is configured to separate solid biological material 72 grown in the at least one flexible production bag 18 from the liquid media containing a pharmaceutical of interest 74 aseptically in an unclassified area. The flexible harvest bag 22 may be disposable. In general, the flexible harvest bag 22 includes three layers of material: a pair of outer layers 68 and a filter 70 positioned between the outer layers (see FIGS. 18b and 18d). The outer layers 68 are made of a flexible material and coupled to one another to define an enclosure 69 therebetween (see FIG. 18b). For example, the outer layers 68 may be coupled to one another about their outer periphery as shown in FIG. 18f to define an enclosure 69 therebetween. The outer layers 68 may comprise an expandable polymeric material such as polyethylene, polypropylene, polyethylene terephtalate glycol (PTEG), polycarbonate, or any appropriately classified and rated polymeric material and may be coupled using any suitable technique such as thermal welding. The flexible harvest bag 22 may be configured to expand to hold the solid biological material 72 such that the solid material does not foul the existing filter 70 or slow the flow of the process stream until the harvest bag has reached its capacity. The filter is selected such that the biological material does not pass through but allows for the passage of the biopharmaceutical.

An inlet 76 may be defined in one of the outer layers 68 and is configured to receive a biological material 72 and a liquid media 74 from the flexible production bag 18 and direct the biological material and liquid media into the enclosure. The flexible harvest bag 22 may include an aseptic connector 24 that is configured to mate with a corresponding aseptic connector 24 associated with the flexible production bag 18 for aseptically transferring the biological material 72 and liquid media 74. Similar to the connection between the flexible seed bag 16 and flexible production bag 18, the connection between the flexible production bags and flexible harvest bags 22 may be temporary or permanent such that the flexible bags 18, 22 may be in temporary or continuous flow communication with one another. The flexible harvest bag 22 may also include an outlet 78 that is defined in one of the outer layers 68 and is configured to transfer the filtered liquid media 74 out of the enclosure 69 while the biological material 72 remains within the enclosure. As shown in FIG. 18d, the inlet 76 and outlet 78 may be defined in one of the outer layers 68. Moreover, an air release valve 77 may be defined in an outer layer 68 opposite the inlet valve as shown in FIG. 18b. The air release valve may be configured to release air from the harvest bag 22 when needed to maintain a desired pressure within the bag.

FIG. 18d also demonstrates that the filter 70 is positioned within the enclosure 69 and between the outer layers 68, wherein the filter is configured to separate the biological material 72 from the liquid media 74. As indicated where the biological material produces a biopharmaceutical that is not secreted into the liquid media, the biological material may be subject to further processing to isolate the biopharmaceutical. Alternatively, where the biopharmaceutical is secreted into the liquid media, the liquid media may be subject to further processing for the isolation of the biopharmaceutical. According to one aspect, one of the outer layers 68 is secured to the filter 70 to define a first pocket 80 within the enclosure for retaining the biological material 72 therein, and the outer layers are secured together to define a second pocket 82 within the enclosure 69 for retaining the liquid media 74 therein (see FIGS. 18b and 18c). Thus, biological material 72 and liquid media 74 transferred through the inlet 76 and into the enclosure 69 will be separated by the filter 70 such that the solid biological material 72 remains in the first pocket 80, while the liquid media passes through the filter and into the second pocket 82 for transfer out of the outlet 78. According to one aspect, the biological material may be biomass or waste, while the liquid media contains the biopharmaceutical of interest to be used for further downstream processing. However, the converse may also be true (i.e., the solid biological material 72 is to be used for downstream processing and the liquid media 74 is biomass). FIG. 18b also shows that a third pocket, or hold reservoir 79, is defined between the outer layers 68. The outlet 78 may be defined in the outer layer 68 including the hold reservoir 79. The hold reservoir 79 may be employed as a flow buffer so that variations in flow rates in and out of the harvest bag 22 due to harvest conditions may be accommodated without undue strain on the harvest bag. Thus, the hold reservoir 79 may be used to ensure that there is no overflow within the harvest bag 22 or that there is too much air within the harvest bag.

The flexible harvest bag 22 may be supported by a frame 84 such that the harvest bag is suspended vertically and separation of the biological material 72 from the liquid media 74 may be facilitated via gravity and/or pumping. In particular, the outer layers 68 may be secured together to define an opening 88 for receiving a support rod 86 therethrough, wherein the support rod is configured to support the flexible harvest bag vertically (see FIG. 18c). The biological material 72 and the liquid media 74 are capable of entering the inlet 76 in an upper portion of one of the outer layers 68 and flowing downwardly through the enclosure 69 such that the liquid media 74 is separated by the filter 70, while the separated liquid media is capable of flowing through the outlet 78 in a lower portion of one of the outer layers 68.

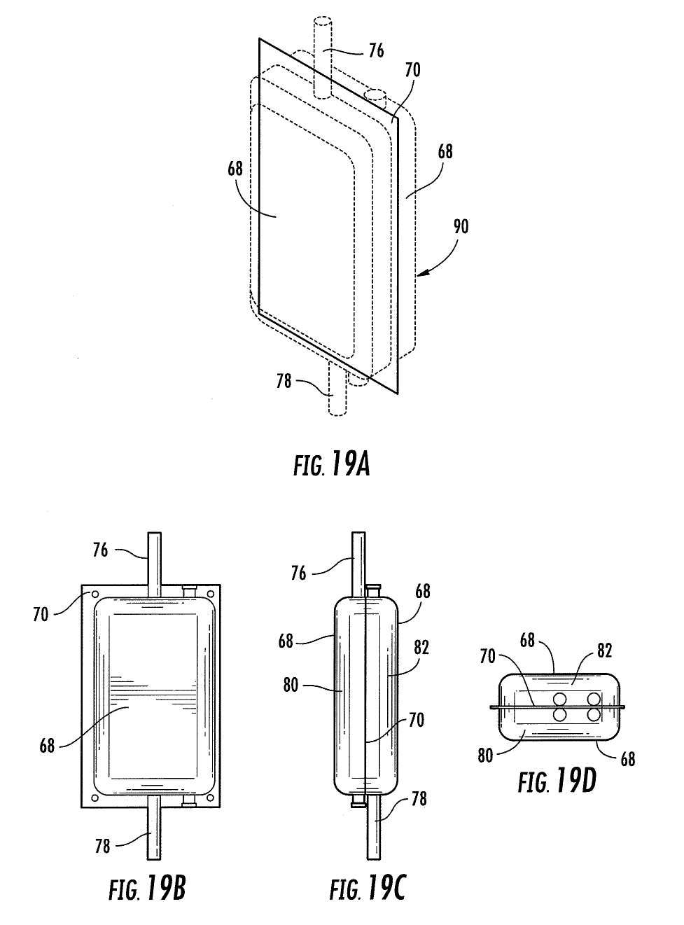

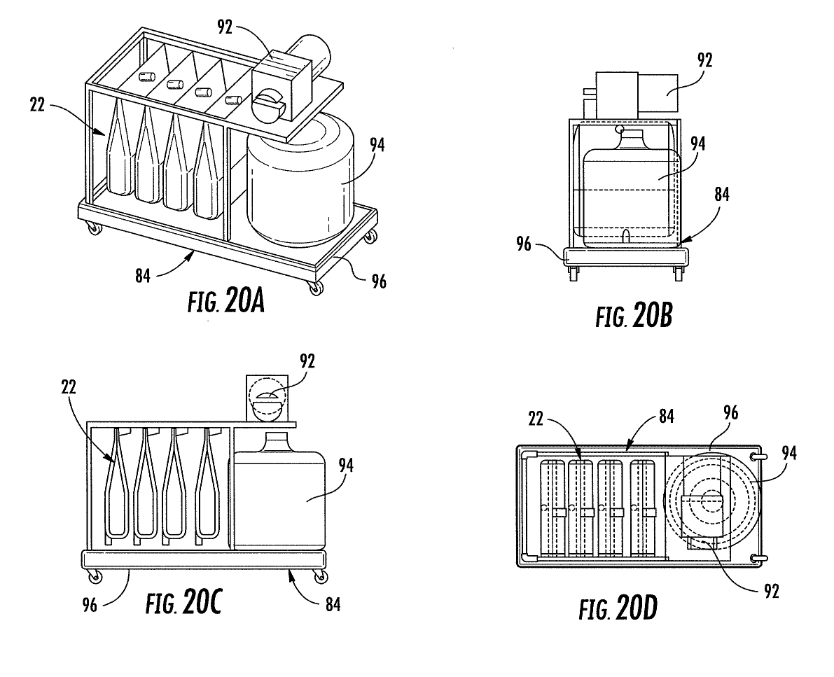

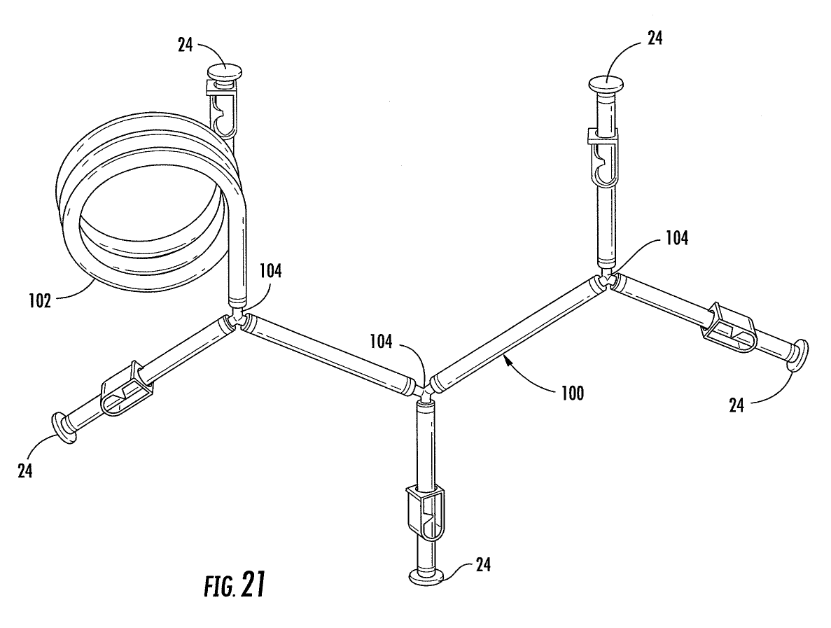

It is understood that the size and configuration of the flexible harvest bags 22 may be modified for particular applications. For example, FIGS. 15-18 show that the harvest bag 22 may have a generally rectangular shape at the inlet end and a generally triangular shape at the outlet end and in the vicinity of the hold reservoir 79. The triangular shape may provide a venture/funnel like geometry to facilitate comprehensive passage of outgoing liquid media to outlet 78. As well, having a hold reservoir 79 below the level of the bottom edge of the filter 70 facilitates complete draining of any captured biological material by gravity. FIGS. 19a-d show an additional embodiment of a harvest bag 90 wherein a filter 70 is similarly positioned between a pair of outer layers 68. However, the filter 70 of the flexible harvest bag 90 completely divides and separates the outer layers 68 as shown in FIGS. 19c and 19d. As before, biological material 72 and liquid media 74 entering the inlet 76 passes into the first pocket 80, and the liquid media is separated from the solid biological filter and enters the second pocket 82 for transfer out of the outlet 78.

In one embodiment, the pore size of the filter 70 provides pre-processing by removing solid biological material in the media of the size specified by the filter pores or gauge. The filter 70 removes at a portion of the solid biological material depending on the pore size of the filter. The tighter or smaller the filter 70 pores the more solid biological material is removed. Prior to downstream media purification, it is desirable to remove as much solid biological material from the media as possible, which is dependent on the pore size of the media filter 70 and the design of the flexible harvest bag 22 which can accommodate small pore filters while still controlling media buildup upstream of the filter by presenting new, unclogged filter material to the media flow as the media level rises in the upstream side of the harvest bag. Thus, the harvest bag 22 can provide a scrubbing application to the media prior to downstream purification because of its self-regulating design.

Furthermore, FIGS. 20a-20d illustrate that a plurality of harvest bags 22 may be supported by a support rack 84 according to one embodiment of the present invention. The support rack 84 may be configured as a mobile cart 96 and include one or more pumps 92 for pumping biological material 72 and liquid media 74 to and from the flexible harvest bags 22, as well as a reservoir 94 for storing the separated liquid media.

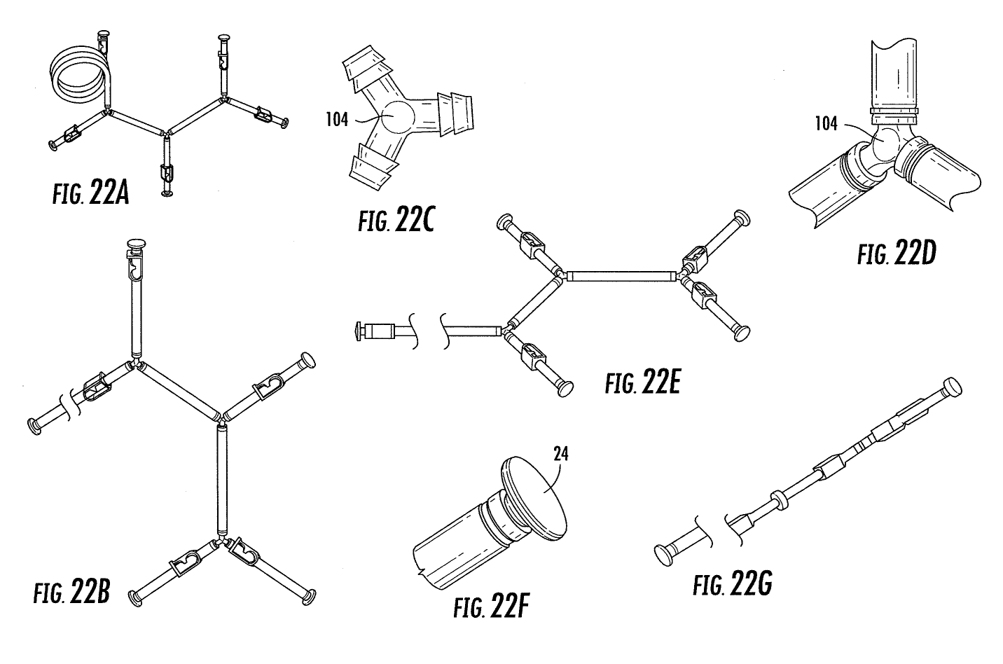

Moreover, FIGS. 22a-g illustrate a harness assembly 100 according to one embodiment of the present invention. The harness assembly 100 includes a plurality of aseptic connectors 24 that are configured to couple with aseptic connectors associated with any one of the flexible seed bags 16, flexible production bags 18, flexible harvest bags 22, or any other container where aseptic transfer of the biological material and liquid media is desired (e.g., carboys). According to one aspect, the harness assembly 100 includes tubing 102 coupled with Y-connectors 104. The harness assembly 100 could be used to couple a plurality of flexible production bags 18 with a single flexible harvest bag 22. For instance, there may be four flexible production bags 18 coupled to the flexible harvest bag 22 with the harness assembly 100.

During use, the culture tubes 12 are initially filled with biological material (e.g., fronds) and liquid media in a classified, aseptic area and sealed with an aseptic connector assembly 14. The biological material may be a surface-borne biological material such as plants from the duckweed family, or the aquatic plant species described above, that require light to proliferate via photosynthesis. The aseptic connector 24 of the culture tube 12 is coupled to the aseptic connector 24 of the flexible seed bag 16 so that the biological material and liquid media may be transferred into the flexible seed bags aseptically. Or, as described above, the culture tube 12 could be connected directly to the flexible production bag 18 such that the flexible seed bag 16 is not used. The flexible seed bags 16 may be filled with liquid media via the media inlet assembly 44 to supply relatively large volumes of the media. As the flexible seed bag 16 is filled it may be monitored either visually, or automatically, to determine at which point the media reaches a level at which a maximized surface area is defined. The flexible seed bags 16 may be arranged on a support rack 20 as described above in a tighter arrangement in order to grow larger amounts of biological material and thereby increase the production density.

After the biological material and media are added, a heat sealer or pinch clamp can seal the culture tube 12 and transfer tube 34 of the flexible seed bag 16, and the culture tube and transfer tube can be cut away leaving the terminated and sealed end of the transfer tube attached to the flexible seed bag. Power may then be supplied to the light source 60 (or the lights may have already been on) so as to cast light through the transparent flexible seed bags 16. Over time, the biological materials draw energy from the light and nutrients from the media and air supply and begin to proliferate. In the case of biological materials used for pharmacological purposes, the biological materials may secrete biopharmaceuticals, including peptides and proteins, into the liquid media. According to one aspect, the biological material proliferates for about 12 to about 30 days, more typically about 21 days, in the flexible seed bags 16.

Also during this time, various properties (e.g., temperature, pH, CO₂ composition, etc.) of the gaseous and media environment in the flexible seed bags 16 may be monitored. In turn, this data is collected and may be used to control the intensity of the light source 60, the temperature and convection properties of the ambient air around the flexible seed bags 16, and the temperature and amounts of gasses and media supplied to the flexible seed bags. In one embodiment, the data that is monitored may be used with a feedback control system 63 to automatically measure and control a temperature of the biological material. In addition, a sampling bag 38 can be used to take small samples to determine the progress of the secretions. Such progress may also be used to determine the various aforementioned conditions within the flexible seed bags 16.

After the biological material has proliferated within the flexible seed bags 16 for a predetermined or otherwise desired time period, the biological material and liquid media may be transferred to the flexible production bags 18. As before, the transfer of biological material and liquid media may occur aseptically via aseptic connectors 24. Similar to the flexible seed bags 16, the flexible production bags 18 also promote proliferation of the biological material therein. In addition, various properties (e.g., temperature, pressure, pH, CO₂ composition, etc.) of the gaseous and media environment in the flexible production bags 18 may be similarly monitored. The flexible production bags 18 may further be positioned in a support rack 20 for increasing the production density of the biological material. According to one aspect, the biological material is allowed to proliferate for about 12 to about 30 days, more typically about 24 days in the flexible production bags 18.

After the biological material has proliferated within the flexible production bags 18 for a desired time period, the biological material and liquid media may be transferred to the flexible harvest bags 22. The transfer of the biological material and liquid media occurs aseptically in an unclassified area using aseptic connectors 24 as described above. The flexible harvest bags 22 are configured to separate the solid biological material 72 (i.e., biomass) from the liquid media 74 (i.e., filtrate). In one embodiment, the liquid media 74 contains active target compounds that may be used for downstream processing and can be pumped out of the flexible harvest bags 22 via the outlet 78. The solid biological material 72 remaining in the flexible harvest bags 22 may be neutralized and discarded. In another embodiment, where the active target compound is tissue bound, the media outlet 78 of the harvest bag 22 can be aseptically connected to the production bag 18 and the filtered media 74 from the harvest bag recirculated through the production bag until all biological material 72 is removed and trapped in the aseptic harvest bag. Once sequestered in the harvest bag 22, the biological material 72 can be aseptically kept until processed. There may be several flexible production bags 18 coupled to a single flexible harvest bag 22 (e.g., via harness 100) such that large scale harvesting of the biological material 72 and liquid media 74 may occur.

Embodiments of the present invention may provide many advantages. Overall, the system 10 allows the production of clinical and commercial scale quantities of biopharmaceuticals from genetically modified plants in a contained, aseptic environment. Thus, no classified or particle controlled areas are required for producing or processing the biological material, and the system 10 is capable of maintaining bioburden free status throughout the processing of the biological material until final purification. In particular, once the culture tubes 12 are capped with an aseptic connector assembly 14, the remaining processing of the biological material and liquid media may occur aseptically in an unclassified area. Thus, no classified areas, tube welding, or special conditions are necessary in order to transfer the biological material aseptically within the system 10. Moreover, each component of the system 10 that contacts the biological material and liquid media may disposable, such as the culture tubes 12, flexible seed bags 16, flexible production bags 18, and flexible harvest bags 18. In addition, the use of flexible bags 16, 18 for partial filling with media provides a relatively large surface for the large-scale production of biopharmaceuticals by surface-borne biological materials, such as duckweed plants, and provides for optimizing proximity to light and air supply for any botanical culture. Moreover, when the flexible bags 16, 18 are used in conjunction with a support rack 20, the production density of the biological material may be increased due to the vertically optimized arrangement and configuration of the flexible bags.

Many modifications and other various embodiments of the invention set forth herein will come to mind to one skilled in the art to which this invention pertains having the benefit of the teachings presented in the foregoing descriptions and the associated drawings. Therefore, it is to be understood that the various embodiments of the invention are not to be limited to the specific embodiments disclosed and that modifications and other embodiments are intended to be included within the scope of the appended claims. Although specific terms are employed herein, they are used in a generic and descriptive sense only and not for purposes of limitation.

1. A bioreactor system for the production and processing of a biological material in an aseptic environment, the system comprising:
at least one culture tube configured to hold a liquid media containing a biological material;
at least one flexible bag for promoting growth of the biological material therein, wherein the at least one culture tube and the at least one flexible bag are configured to transfer liquid media and biological material from the at least one culture tube to the at least one flexible bag aseptically in an unclassified area; and
at least one flexible harvest bag configured to be in flow communication with the at least one flexible bag, the at least one flexible harvest bag configured to separate biological material grown in the at least one flexible bag from the liquid media aseptically in an unclassified area.

2. The system of claim 1, wherein the at least one culture tube and the at least one flexible bag comprise an aseptic connector configured to couple to one another and facilitate aseptic transfer of the biological material and liquid media.

3. The system of claim 1, wherein the at least one flexible bag comprises a flexible seed bag or a flexible production bag.

4. The system of claim 1, wherein a height of the at least one flexible bag is substantially less than a length and width thereof.

5. The system of claim 1, further comprising a plurality of flexible bags.

6. The system of claim 5, wherein the plurality of flexible bags are configured to be in flow communication with a single harvest bag.

7. The system of claim 5, further comprising a support rack configured to support the plurality of flexible bags spaced apart vertically from one another.

8. The system of claim 7, wherein the support rack further comprises a feedback control system for automatically measuring and controlling a temperature of the biological material in the plurality of flexible bags.

9. The system of claim 7, wherein the support rack further comprises:
a plurality of upright support members;
a plurality of laterally-extending support rails interconnecting the upright support members; and
a plurality of shelves engaged with the laterally-extending support rails, the shelves being configured to support the plurality of flexible bags spaced apart vertically from one another.

10. The system of claim 11, further comprising a plurality of light sources disposed between the plurality of shelves.

11. The system of claim 1, further comprising a culture tube assembly comprising:
a culture tube configured to hold an agar-based media or a liquid media containing a biological material; and
an aseptic connector assembly coupled to the culture tube and configured to aseptically contain the media and biological material within the culture tube, the aseptic connector assembly comprising tubing for coupling with the culture tube, the aseptic connector assembly further comprising an aseptic connector configured to transfer the media containing the biological material aseptically in an unclassified area to the at least one flexible bag for promoting growth of the biological material.

12. A method for the production and processing a biological material in an aseptic environment, the method comprising:
transferring a liquid media containing a biological material from at least one culture tube to at least one flexible bag;
promoting growth of the biological material within the at least one flexible bag; and
separating the biological material grown in the at least one flexible bag from the liquid media,
wherein each of the transferring, promoting, and separating steps occurs aseptically in an unclassified area.

13. The method of claim 12, wherein promoting comprises exposing the at least one flexible bag to a light source so as to promote growth of the biological material via photosynthesis.

14. The method of claim 12, further comprising aseptically transferring the biological material and liquid media from the at least one flexible bag to at least one harvest bag, wherein separating comprises filtering the biological material from the liquid media in the at least one harvest bag.

15. The method of claim 12, wherein transferring comprises coupling the at least one culture tube and the at least one flexible bag with respective aseptic connectors.

16. A method of claim 12, further comprising positioning a plurality of the flexible bags in a support rack spaced apart vertically from one another.

17. The method of claim 12, further comprising automatically measuring and controlling a temperature of the biological material in the at least one flexible bag.

18. The method of claim 12, wherein promoting comprises promoting growth of the biological material such that the biological material produces and secretes a biopharmaceutical into the liquid media.

19. A flexible harvest bag assembly for harvesting a solid biological material aseptically in an unclassified area, the flexible harvest bag assembly comprising:
a flexible bag defining an enclosure therein;
an inlet defined in the flexible bag and configured to receive a solid biological material and a liquid media and direct the biological material and liquid media into the enclosure;
a filter positioned within the enclosure, the filter configured to separate at least a portion of the solid biological material from the liquid media; and
an outlet defined in the flexible bag and configured to transfer the filtered liquid media out of the enclosure while the solid biological material remains within the enclosure.

20. The flexible harvest bag assembly of claim 19, wherein the flexible bag comprises a pair of outer layers of flexible material coupled to one another to define an enclosure therebetween.

21. The flexible harvest bag assembly of claim 20, wherein the pair of outer layers comprise an expandable polymeric material.

22. The flexible harvest bag assembly of claim 20, wherein the inlet and the outlet are defined in one of the outer layers of flexible material.

23. The flexible harvest bag assembly of claim 22, wherein the inlet and outlet are located at opposite ends of the same outer layer of flexible material.

24. The flexible harvest bag assembly of claim 22, further comprising an air release valve defined opposite the inlet in the other outer layer of flexible material.

25. The flexible harvest bag assembly of claim 20, wherein the filter is positioned within the enclosure and between the outer layers of flexible material.

26. The flexible harvest bag assembly of claim 25, wherein one of the outer layers of flexible material is secured to the filter to define a first pocket within the enclosure for retaining the solid biological material therein, and wherein the outer layers of flexible material are secured together to define a second pocket within the enclosure for retaining the liquid media therein.

27. The flexible harvest bag assembly of claim 26, wherein the second pocket comprises a hold reservoir for buffering inconsistencies in flow rate through the inlet and the outlet due to harvesting conditions without causing over-pressure within the flexible bag.

28. The flexible harvest bag assembly of claim 20, wherein the outer layers of flexible material are secured together to define an opening for receiving a support rod therethrough, the support rod configured to support the flexible bag vertically such that the solid biological material and the liquid media are capable of entering the inlet in an upper portion of the one of the outer layers and the liquid media is capable of exiting the outlet in a lower portion of one of the outer layers.

29. The flexible harvest bag assembly of claim 19, wherein the flexible bag has a generally rectangular shape proximate the inlet and a generally triangular shape proximate the outlet.

30. The flexible harvest bag assembly of claim 19, wherein the inlet is configured to receive a solid biological material and a liquid media containing a biological material and/or a biopharmaceutical and direct the solid biological material and liquid media containing the biological material and/or biopharmaceutical into the enclosure, wherein the filter is configured to separate at least a portion of the solid biological material from the liquid media containing the biological material and/or biopharmaceutical, and wherein the outlet is configured to transfer the filtered liquid media containing the biological material and/or biopharmaceutical out of the enclosure while the solid biological material remains within the enclosure.
